(19) United States
(12) Patent Application Publication
(10) Pub. No.: US 2026/0306315 A1
(43) Pub. Date: Oct. 8, 2026

(54) TEMPERING DEVICE FOR CONTROLLING THE TEMPERATURE OF A LIVING BEING

(71) Applicant: KOHLBRAT & BUNZ Gesellschaft m.b.H., Radstadt (AT)

(72) Inventor: Michael GRAF, Zederhaus (AT)

(21) Appl. No.: 19/638,825

(22) Filed: Apr. 3, 2026

(30) Foreign Application Priority Data
Apr. 4, 2025 (AT) A 50240/2025

(51) Int. Cl. A61G 1/01; A61G 1/048; F25B 21/04

(52) U.S. Cl. CPC A61G 1/01; A61G 1/048; F25B 21/04; A61G 2210/70; A61G 2210/90; F25B 2321/0251; F25B 2321/0252

(57) ABSTRACT

A temperature control device for temperature control of a living being, in particular a human being, comprising a shell with a first side for supporting a living being, and at least one temperature control element which is arranged inside the shell, wherein a heat flow can be generated by means of the at least one temperature control element, wherein the temperature control device comprises at least one heat transfer element arranged inside the shell, by means of which the heat flow generated by the at least one temperature control element can be transferred to the first side of the shell in order to heat and/or cool a living being located on the temperature control device.

The present invention relates to a temperature control device for temperature control of a living being, in particular a human being, comprising a shell with a first side for supporting a living being, and at least one temperature control element which is arranged inside the shell, wherein a heat flow can be generated by means of the at least one temperature control element. Furthermore, the invention also relates to a device with such a heating element and an arrangement consisting of such a heating element and a support device for a living being.

In physical terms, heat flow is understood as the amount of heat that is transferred from a place of high temperature to a place of low temperature. Depending on the direction of the heat flow, the heat flow generated by the temperature control element can therefore result in either warming or cooling.

Temperature control devices of this type are already sufficiently known from the prior art, for example from DE 10 2015 007 526. This document discloses a temperature-regulating support pad with a shell. Directly beneath a PVC top layer, onto which a living being can lie, a temperature control element in the form of a carbon heating mat is arranged, which can be used to warm the living being on the support. The carbon heating mat extends over a large part of the surface of the temperature control device.

A disadvantage of such temperature control devices is that the temperature control element arranged in the shell cannot be used to cool a living being. In addition, large-area temperature control elements are necessary to adequately regulate the temperature of the living being lying on the support. However, such large-area temperature control elements can be costly and also prone to damage, which is why the aforementioned DE 10 2015 007 526 provides for an additional stabilizing layer for the temperature control element.

Furthermore, it is not possible to evacuate the interior of a temperature control device according to DE 10 2015 007 526, as it has a shell that can be opened via a zipper. However, this can bring some advantages, which will be described in more detail below.

The object of the present invention is to provide a temperature control device that is improved compared to the prior art and that at least partially avoids the aforementioned disadvantages, as well as a device with such a heating element and an arrangement consisting of such a heating element and a support device for a living being.

These objects are achieved by the features of claims 1, 14 and 15.

According to the invention, in the above temperature control device it is provided that the temperature control device comprises at least one heat transfer element arranged within the shell, by means of which the heat flow generated by the at least one temperature control element can be transferred to the first side of the shell in order to warm and/or cool a living being located on the temperature control device.

This makes it possible to provide a heat flow via the temperature control element, which has a warming and/or cooling effect on the first side of the shell. Accordingly, a living being located on the shell can be warmed and/or cooled.

A heat transfer element according to the invention has the advantage that a large-area temperature control element is not required. This allows for the use of small temperature control elements, which are cost-effective and less susceptible to damage. An additional stabilizing layer can also be omitted.

Regarding a cooling temperature control element, a heat transfer element according to the invention allows for a meaningful implementation of a temperature control device with sufficient cooling capacity in the first place. Flat temperature control elements that can produce a cooling effect and are suitable for use in temperature control devices of this type are either unknown or too expensive.

With regard to a device according to the invention, it is provided that the temperature control device is arranged within the device. The temperature control device is therefore integrated into the device, resulting in easy handling of the device.

In an arrangement according to the invention, the temperature control device is arranged on a side of the support device facing the living being. The temperature control device thus serves as a support between a living being and the support device.

Further preferred embodiments are defined in the dependent claims.

The heat transfer element can be connected to the temperature control element to achieve simple heat transfer from the temperature control element to the heat transfer element. This heat transfer can preferably occur via heat conduction and/or heat radiation.

According to one exemplary embodiment of the invention, the shell may have at least two planar material webs, between which at least one interior space is formed, wherein the at least two material webs are connected to each other, preferably permanently, at least along their respective outer edges, preferably by welding.

This represents a simple and cost-effective way to implement a shell according to the invention. Especially when the two material webs are permanently joined at their outer edges, a closed shell can be easily provided.

In other words, the shell can be free of openings that would allow the removal of the at least one heating element and the at least one energy source. The shell can therefore be substantially completely closed, with the exception of smaller openings for, for example, a valve for evacuating an interior of the shell or an interface for an external energy source.

This makes it possible to provide a shell that is watertight, gas-tight, and evacuable, as no openings or the like need to be provided for the possible removal of the temperature control element or any other component located inside the shell.

The material webs can be made of plastic or similar materials. It is also conceivable that the material webs consist of a composite material, for example coated fabric.

In any case, the materials should be suitable for use in the medical field and/or be watertight and gas-tight.

Advantageously, it can be provided that the at least one transfer element is arranged directly on a first material web of the at least two material webs, wherein the first material web forms the first side of the at least one shell.

Thus, only one of the material layers is located between the heating element and a living being, ensuring good heat transfer from the temperature control element to the living being or vice versa.

It may also be provided that the shell is waterproof and/or gas-tight and/or elastic.

A water- and gas-tight design of the shell allows for easy and thorough cleaning of the shell. Furthermore, components inside the shell are protected as effectively as possible from harmful environmental influences.

An elastic structure of the shell allows it to adapt well to the shape of a living being. In this respect, it can also be advantageous to provide that the heat transfer element is elastically designed. In some embodiments, the temperature control element can also be elastically designed, even though this is not necessary due to the heat transfer element.

Preferably, the temperature control element may comprise at least one Peltier element, preferably be formed by at least one Peltier element.

A Peltier element generates a temperature difference and thus a heat flow when an electric current flows through it. In other words, when an electric current flows through it, one side of the Peltier element cools down while another side of the Peltier element heats up.

Accordingly, the Peltier element can be used for both cooling and heating a living being located on the temperature control device, depending on which side of the Peltier element the heat flow is taken from by the heat conducting element. The Peltier element can therefore fulfill a dual function. Furthermore, Peltier elements are small in size, light in weight and, in the size required for a typical temperature control device, inexpensive.

This makes it easy to arrange Peltier elements within the shell of a temperature control device, which would not be possible with conventional refrigeration machines. This is particularly advantageous because no external devices (possibly with the exception of a power supply) are necessary to operate the temperature control device.

With regard to a Peltier element, it may also be provided that at least one heat transfer element is connected and/or connectable to a first side and/or a second side of the Peltier element.

It is also conceivable that the heat transfer element is connected to the first or second side of the Peltier element, depending on whether a living being is to be cooled or warmed.

This allows the dual function of the Peltier element to be utilized.

According to a further embodiment of the invention, at least two temperature control elements can be provided, wherein a first of the two temperature control elements is responsible for heating a living being and the other of the two temperature control elements is responsible for cooling a living being.

This allows the most suitable temperature control element to be selected for both cooling and heating.

If both temperature control elements are designed as Peltier elements, it can be provided that a first of the two Peltier elements is in contact with the heat transfer element with a first side and the second of the two Peltier elements is in contact with the heat transfer element with a second side. This allows cooling to be performed using a first Peltier element and heating using a second Peltier element.

It is also conceivable that two heat transfer elements are provided for two temperature control elements.

Preferably, it can be provided that the heat transfer element comprises
an airtight upper layer,
an airtight lower layer opposite the upper layer,
at least one airtight intermediate layer arranged between the upper layer and the lower layer, and
a transport means for transporting a gas, preferably air, through the intermediate layer
wherein the heat flow generated by the at least one temperature control element can be transferred to the first side of the shell via the gas transportable through the intermediate layer.

This represents an advantageous possibility for implementing a heat transfer element. The temperature control element releases heat to a gas or removes heat from the gas (“releases cold”). The gas heated or cooled by the temperature control element is then transported via the transport means through the intermediate layer to the first side of the shell, where it releases heat to the first side or extracts heat from it, preferably via heat conduction between the transfer element and the first side.

The transport means can, for example, include a fan.

The gas is preferably air. However, it is also conceivable that other gases with better thermal conductivity could be used.

It may also be provided that the shell has an inlet opening for air to enter the shell and an outlet opening for air to exit the shell.

It may be provided that air is conveyed via the transport means through the inlet opening into the shell, through the heat transfer element and then through the outlet opening out of the shell.

This makes it possible to remove “used” air, which has released its heat to the first side or absorbed heat from the first side, from the temperature control device. At the same time, “fresh” ambient air can be supplied, which can then be heated and/or cooled via the temperature control element.

However, it is also conceivable that the gas is in a closed circuit, wherein the gas that has released its heat to the first side or absorbed heat from the first side is fed back to the temperature control element for heating or cooling. This is particularly interesting if the gas is not air.

Preferably, the upper layer, the lower layer and the intermediate layer may form a spacer fabric.

This allows the heat transfer element to also act as padding for the living being, thus increasing the living being comfort while lying down. The heat transfer element therefore has a dual function. According to a further embodiment, it can be provided that the at least one heat transfer element is connected to the first side and the at least one temperature control element, so that the heat flow generated by the at least one temperature control element can be transferred by means of heat conduction to the at least one heat transfer element and from the at least one heat transfer element to the first side of the shell.

This represents a simple possibility for implementing a heat transfer element. In particular, the heat transfer element in this embodiment can be designed as a film.

Advantageously, it can be provided that the heat transfer element has a thermal conductivity coefficient of at least 1 W/mK. Of course, higher thermal conductivity coefficients can also be used, since the efficiency of heat conduction also increases with increasing thermal conductivity coefficients.

It may be provided that at least one heat transfer element comprises a thermally conductive gel and/or a thermally conductive liquid and/or a metal.

If the heat transfer element comprises a thermally conductive gel and/or a thermally conductive liquid, it may comprise an enclosure in which the thermally conductive gel and/or the thermally conductive liquid is arranged.

The enclosure itself can also be thermally conductive.

The temperature control element can be in direct contact with the thermally conductive gel and/or the thermally conductive liquid or with the enclosure.

Preferably, the at least one heat transfer element extends over at least 30%, preferably over at least 50%, of the surface of the shell and/or the at least one temperature control element extends over a maximum of 25% of the surface of the shell.

The heat transfer element can, in particular, also extend over at least 60%, 70%, 80%, 85%, 90%, 95% or 100% of the area.

The heat transfer element can also have an area, wherein the area of the heat transfer element is at least 30%, 50%, 60%, 70%, 80%, 85%, 90%, 95% or 100% of the area of the shell.

This ensures that a living being located on the temperature control device can be adequately and evenly tempered at all points.

The temperature control element can also extend over a maximum of 20%, 15%, 10% or 5% of the area of the shell.

The temperature control element does not need to have a large surface area, but can also be relatively small. This allows for a smaller temperature control element that is less prone to damage.

Furthermore, it may also be provided that the heating element has insulation, preferably in the form of granules and/or a spacer fabric or woven material.

The insulation serves, on the one hand, to prevent unwanted heat radiation in a direction in which no living being is in contact with the heat transfer element or the first side of the shell. On the other hand, insulation can also serve to stabilize the heat transfer element, although this is not mandatory.

Particularly preferably, it may be provided that vacuum can be created in at least one interior space of the shell, preferably via a valve arranged on the shell. In other words, a vacuum can be created inside the shell.

This results in some advantages over conventional temperature control devices. On the one hand, the heat transfer element is substantially fully formed on the shell due to the evacuation of the interior, so that good heat transfer between the heat transfer element and the shell, and subsequently between the shell and the living being, is ensured.

On the other hand, this allows the heat transfer element to be well suited to the contour of a living being (for example, head or shoulder). This ensures the greatest possible direct contact of the heat transfer element with the living being, thereby enabling effective heating of the living organism. This effect is further enhanced if insulation is provided, which prevents or reduces the radiation of heat away from a living being. Furthermore, the shaping of the heat transfer element on the living organism leads to a uniform pressure distribution in the region of the temperature control device, thus preventing pressure points.

The shaping process can be done analogously to vacuum mattresses. This means that the living being is placed on the non-evacuated temperature control device, which then adapts to the contour of the living being via the insulation in the form of granules or a spacer fabric or material. When the temperature control device is evacuated, the temperature control device is then fixed in this adapted shape by the insulation.

Advantageously, a corresponding valve is provided for evacuation, through which the interior of the shell can also be ventilated again. The vacuum can be generated using a device that is already well known.

Advantageously, it can be provided that the temperature control device comprises at least one interface for supplying the at least one temperature control element with energy, preferably electrical energy, from an external energy source.

The interface can be located in an opening in the shell or led out of the shell. It is also conceivable that the interface is located inside the shell.

The shell and the interface can be designed in such a way that the interior of the shell can still be evacuated.

The interface can be, for example, a plug or a socket. However, the interface can also be designed in the form of an inductive interface for the inductive transfer of energy.

An external energy source is an energy source that is separate from and does not belong to the temperature control device. This could be, for example, a conventional electrical outlet, a power connection in a vehicle, or a mobile battery.

According to a further exemplary embodiment, it may be provided that the temperature control device has at least one temperature sensor, wherein the temperature control element can be regulated depending on measured values of the at least one temperature sensor.

This ensures that the living being is always ideally tempered via the temperature control device.

Particularly preferred is at least one control device for controlling an energy supplied to the at least one temperature control element, preferably electrical energy, depending on measured values of the at least one temperature sensor.

Advantageously, the control device can be designed to regulate the energy supplied to the at least one temperature control element, preferably electrical energy, in such a way that a predefined temperature is not exceeded and/or fallen below at the heat transfer element.

When using a temperature control device, it is important that no damage such as burns is caused to the living being by the application of heat. It is therefore important to regulate the maximum temperature on the temperature control device. The relevant literature states that the maximum skin contact temperature for humans to avoid burns is 60° C. for a duration of 5 seconds. However, direct contact with the skin or tissue can cause cell damage even at temperatures as low as 45° C. It can therefore be advantageous to limit the temperature to below 45° C. Based on clinical studies, a maximum temperature of 42° C. is desirable.

It can also be provided that the temperature sensor is located on the first side of the shell and/or that the insulation is located on a second side of the shell opposite the first side. This second side faces away from the living being.

As already explained, this makes it possible to ensure that the largest possible portion of the heat or cold generated by the temperature control element can be supplied to the living being as directly as possible. This allows losses to be minimized and the efficiency of the temperature control device to be increased.

It may also be provided that the temperature control device has an operating element for operating the temperature control device. The operating element can be used, for example, to switch the temperature control device on or off, or to set a target temperature value to which a living being is to be heated or cooled.

It may also be provided that the temperature control device has at least one fastening element for fastening the temperature control device to a support device for a living being, in particular a stretcher, that is separate from the temperature control device.

This makes it easy to fasten the temperature control device to a support device. The at least one fastening element could be, for example, eyelets, straps, or other suitable means.

A temperature control device according to the invention can be used in a variety of ways, for example as a pillow, heating pad or seat cushion. The temperature control device can also be integrated into such products. Furthermore, vacuum mattresses and neck braces with integrated temperature control devices are conceivable.

Further details and advantages of preferred embodiments of the invention are explained in more detail hereinafter based on the description of the figures with reference to the drawings. In particular:

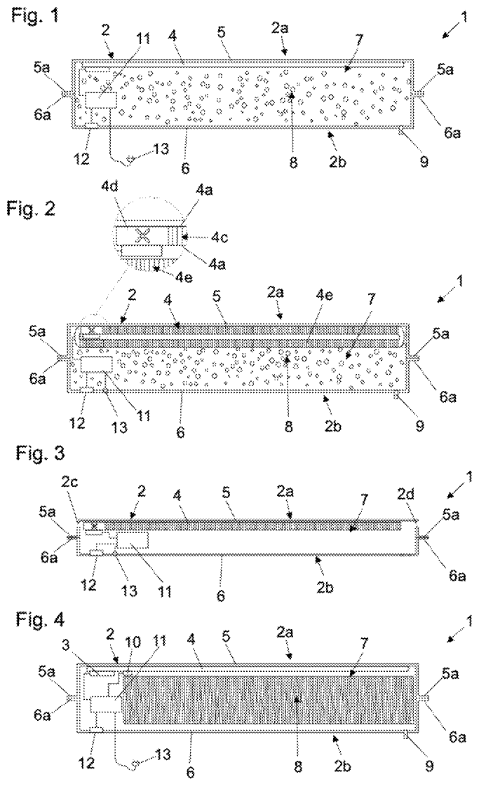

FIG. 1 shows a schematic sectional view of an exemplary embodiment of a temperature control device,

FIG. 2 shows a schematic sectional view of a further exemplary embodiment of the temperature control device,

FIG. 3 shows a schematic sectional view of a further exemplary embodiment of the temperature control device,

FIG. 4 shows a schematic sectional view of a further exemplary embodiment of the temperature control device,

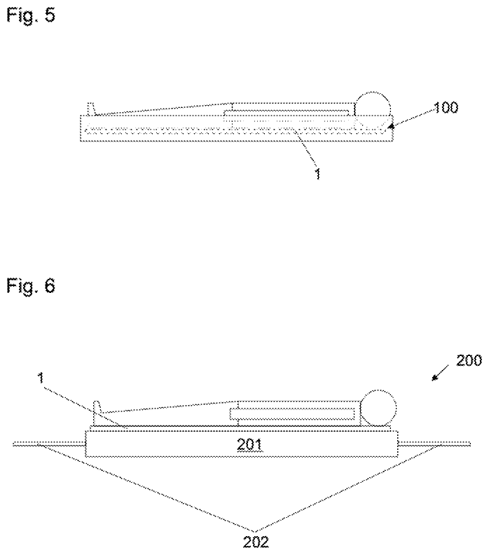

FIG. 5 shows a schematic view of an exemplary embodiment of a device, and

FIG. 6 shows a schematic view of an exemplary embodiment of an arrangement.

FIG. 1 shows a schematic sectional view of an exemplary embodiment of a temperature control device 1. A shell 2 is visible, wherein the shell 2 has a first material web 5 and a second material 6, which are inseparably connected to each other along their outer edges 5a, 6a. In the present embodiment, this inseparable connection can be created by welding the two material webs 5, 6 together. The shell 2 has a first side 2a and a second side 2b opposite the first side. The first side 2a is a side to be turned towards a living being and the second side 2b is a side to be turned away from a living being.

The shell 2 forms an interior space 7 within which the components of the temperature control device 1 are arranged. The temperature control device 1 can thus be designed as a self-contained system, which is protected from environmental influences due to the water-and gas-tight design of the shell 2.

A connection to the interior space 7 can be established via valve 9 in order to evacuate it or to ventilate it again after evacuation.

In addition, in this exemplary embodiment, an interface 13 in the form of a cable with a plug 13 is brought out of the shell 2. An operating element 12 is integrated into the second material web 6 of the shell 2. However, this has no effect on the aforementioned properties of shell 2.

Inside the interior space 7, a temperature control element 3, a heat transfer element 4 and a control device 11 for regulating or controlling the temperature control element 3 can also be seen.

The heat transfer element 4 is arranged directly on the first material web 5, and thus on the first side 2a, in order to enable the best possible heat transfer from the heat transfer element 4 to a living being.

In this embodiment, the heat transfer element 4 is, for example, designed as a film or comprises a conductive gel or liquid enclosed in an enclosure.

In this embodiment, the temperature control element 3 is connected to the heat transfer element 4 and thus transfers heat or cold by means of heat conduction to the heat transfer element 4, which in turn is in contact with the first material web 2a.

It can be seen that the temperature control element 3 can be small, but the heat transfer element 4 extends over a large part of the surface area of the shell 2 or the first material web 5.

Furthermore, insulation 8 in the form of granules is visible, which at least partially fills the interior space 7. The granules can, for example, consist of polystyrene. The insulation 8 reduces heat transfer from the heat transfer element 3 towards the second side 2b, thus making more heat or cold available on the first side 2a.

The interior space 7 can be evacuated via valve 9. The insulation 8 allows the heating element 1 to be shaped according to the contours of a living being, similar to a vacuum mattress.

The interface 13 is connected to the control device 10. The control device 10 is connected to the operating element 12 and the temperature control element 3. The control device 10 can supply energy to the temperature control element 3 and control and/or regulate it, for example via user inputs via the operating element 12.

FIG. 2 shows a schematic sectional view of another exemplary embodiment of a temperature control device 1. This differs from the exemplary embodiment according to FIG. 1 substantially in the design of the heat transfer element 4 and the interface 13.

The heat transfer element 4 comprises an airtight upper layer 4a, an airtight lower layer 4b and an air-permeable intermediate layer 4c. Furthermore, a transport means 4d for transporting a gas, preferably air, is recognizable through the intermediate layer 4c.

The temperature control element 3 is connected to the transport means 4 and releases heat to it or removes heat from it. A gas can be transported through the heat transfer element 4 via a fan of the transport means 4d, thus heating or cooling the first material web 5 which is in contact with the heat transfer element 4.

In the present exemplary embodiment, a closed system is provided. This means that after a gas has transferred heat to the first material web 5 via the heat transfer element or extracted heat from it, it can be fed back to the temperature control element 3 via a return line 4e and heated or cooled therein.

In this exemplary embodiment, the upper layer 4a, the lower layer 4b and the intermediate layer form a spacer fabric.

The return line 4e is designed analogously to the heat transfer element 4. However, a different design for the return line 4e may also be provided.

In this exemplary embodiment, the interface 13 is designed not as a plug, but as a socket integrated into the shell.

FIG. 3 shows a schematic sectional view of another embodiment of a temperature control device 1. This differs from the exemplary embodiment according to FIG. 2 substantially in that an open system is provided for the heat transfer element 4.

The shell 2 therefore has an inlet opening 2c for air to enter the interior space 7 and an outlet opening 2d for air to exit the interior space 7.

Air can therefore be transported into the shell 2 and into the heat transfer element 4 via the transport means 4d and then conveyed out of the shell 2 again via the outlet opening 4d. Therefore, no return line 4e is provided.

A temperature control device 1 according to this exemplary embodiment can therefore not be evacuated. Therefore, no insulation 8 in the form of granules is provided. However, it is conceivable that an insulation 8 is designed in the form of a spacer fabric or woven material, similar to the following exemplary embodiment shown in FIG. 4.

FIG. 4 shows a schematic sectional view of another exemplary embodiment of a temperature control device 1. This differs from the exemplary embodiment according to FIG. 1 substantially in that the insulation 8 is designed in the form of a spacer fabric or woven fabric. In addition, a temperature sensor 10 is also visible, which can measure the temperature at the heat transfer element 4. Based on these measured values, the control device 10 can then regulate the energy supplied to the heating element 3 in order to maintain or not exceed a certain temperature.

However, such a temperature sensor 4 can be provided in each of the illustrated exemplary embodiments.

FIG. 5 shows a schematic view of an exemplary embodiment of a device 100. The device 100 designed as a vacuum mattress. It is evident that a person is accommodated inside the vacuum mattress. In addition, a heating element 1 is indicated, which is arranged within the device 100 and through which heat can be supplied to the person in the device 100.

FIG. 6 shows a schematic view of an exemplary embodiment of an arrangement 200. A support device 201 can, for example, be designed as a stretcher and have carrying handles 202. A heating element 1 is arranged on the support device 201, on which a person can lie. Heat can be supplied the person on the support device 200 via the heating element 1.

### LIST OF REFERENCE NUMERALS

1 temperature control element
2 shell
2a first side
2b second side
2c inlet opening
2d outlet opening
3 temperature control element
4 heat transfer element
4a upper layer
4b lower layer
4c intermediate layer
4d transport means
4e return line
5 first material web
5a outer edge
6 second material web
6a outer edge
6b viewing window
7 interior space
8 insulation
9 valve
10 temperature sensor
11 control device
12 operating element
13 interface
100 device
200 arrangement
201 support device
202 carrying handles

1. A temperature control device for temperature control of a living being, in particular a human being, comprising
a shell with a first side for the support of a living being, and
at least one temperature control element, which is arranged within the shell, wherein a heat flow can be generated by means of the at least one temperature control element,
wherein the temperature control device comprises at least one heat transfer element arranged within the shell, by means of which the heat flow generated by the at least one temperature control element can be transferred to the first side of the shell in order to warm and/or cool a living being located on the temperature control device.

2. The temperature control device according to claim 1, wherein the shell has at least two planar material webs, between which at least one interior space is formed, wherein the at least two material webs, are connected, preferably welded, to each other at least along their respective outer edges preferably inseparably.

3. The temperature control device according to claim 2, wherein the at least one heat transfer element is arranged directly on a first material web of the at least two material webs wherein the first material we forms the first side of the at least one shell.

4. The temperature control device according to claim 1, wherein the shell is waterproof and/or gas-tight and/or elastic.

5. The temperature control device according to claim 1, wherein the temperature control element comprises at least one Peltier element, preferably formed by at least one Peltier element.

6. The temperature control device according to claim 1, wherein the heat transfer element comprises
an airtight upper layer,
an airtight lower layer opposite the upper layer,
at least one airtight intermediate layer arranged between the upper layer and the lower layer, and
a transport means for transporting a gas, preferably air, through the intermediate layer
wherein the heat flow generated by the at least one temperature control element can be transferred to the first side of the shell via the gas transportable through the intermediate layer.

7. The temperature control device according to claim 6, wherein the upper layer, the lower layer and the intermediate layer form a spacer fabric.

8. The temperature control device according to claim 1, wherein the at least one heat transfer element is connected to the first side and the at least one temperature control element so that the heat flow generated by the at least one temperature control element can be transferred by means of heat conduction to the at least one heat transfer element and from the at least one heat transfer element to the first side of the shell.

9. The temperature control device according to claim 8, wherein the heat transfer element has a thermal conductivity coefficient of at least 1 W/mK.

10. The temperature control device according to claim 9, wherein the at least one heat transfer element comprises a thermally conductive gel and/or a thermally conductive liquid and/or a metal.

11. The temperature control device according to claim 1, wherein the at least one heat transfer element extends over at least 30%, preferably over at least 50%, of the area of the shell and/or wherein the at least one temperature control element extends over a maximum of 25% of the area of the shell.

12. The temperature control device according to claim 1, wherein at least one interior space of the shell can be evacuated, preferably via a valve arranged on the shell.

13. The temperature control device according to claim 1, wherein the temperature control device comprises at least one interface for supplying the at least one temperature control element with energy, preferably electrical energy, from an external energy source.

14. A device in particular a vacuum mattress, for supporting and/or stabilizing at least one living being, in particular a human being, with a temperature control device according to claim 1, wherein the temperature control device is arranged within the device.

15. An arrangement comprising a support device for a living being, in particular a stretcher, and a temperature control device according to claim 1, wherein the heating element is arranged on a side of the support device facing the living being.
